(19) United States
(12) Patent Application Publication
(10) Pub. No.: US 2025/0273332 A1
(43) Pub. Date: Aug. 28, 2025

(54) Process and Evaluation Method to Identify Signs of a Potential Concussion in Active Children and Others called Safe Brain.

(71) Applicants: Bob Ford, Louisville, KY (US); Jonathan Rowland, Louisville, KY (US)

(72) Inventors: Bob Ford, Louisville, KY (US); Jonathan Rowland, Louisville, KY (US)

(21) Appl. No.: 18/586,495

(22) Filed: Feb. 25, 2024

(51) Int. Cl. G16H 50/20; G16H 10/20; G16H 10/60; G16H 15/00; G16H 80/00

(52) U.S. Cl. CPC G16H 50/20; G16H 10/20; G16H 10/60; G16H 15/00; G16H 80/00

(57) ABSTRACT

An Evaluation Method to Identify Signs of a Potential Concussion in Active Children called Safe Brain. The method is a neurological evaluation method to assess changes in cognitive function and brain performance over time to reveal changes in the cognitive status of a person and helping identify signs of potential head injury wherein changes exist. This method uses a mobile app that alerts children and caregivers that a change may have transpired. Safe Brain has designed and developed a full method of application and games that establish baseline levels for comparisons using a mobile phone. Caregivers register the application and individual neurological baseline values are established for each account of the child. Once set up, Safe Brain continues to creatively engage with children and caregivers which provides more objective criteria that indicates potential symptoms of a concussion indicated by changes from the baseline values.

### CROSS-REFERENCE TO RELATED APPLICATIONS

This application claims the benefit of United States Provisional Patent Application with Ser. No. 63/448,490 filed Feb. 27, 2023, by applicants Bob Ford and Jonathan Rowland. The application was entitled “A Process and Evaluation Method to Identify Signs of a Potential Concussion in Active Children and Others called Safe Brain.”

### FIELD OF INVENTION

This invention relates to a neurological evaluation method and process to assess cognitive function and measure brain performance over time. This evaluation method has the goal and/or the purpose of revealing a change in the cognitive status of a person and helping to identify signs that one may have been subjected to head injury such as a concussion.

### FEDERALLY SPONSORED RESEARCH

None.

### SEQUENCE LISTING OR PROGRAM

None.

### BACKGROUND-FIELD OF INVENTION and PRIOR ART

### Background

Supported by a novelty search of this process and evaluation method which the Applicants entitled “Process and Evaluation Method to Identify Signs of a Potential Concussion in Active Children and Others called Safe Brain,” it is believed, as far as known and researched, that there are no other methods and processes like the “Process and Evaluation Method to Identify Signs of a Potential Concussion called Safe Brain or the like. It is believed that this evaluation process is unique in its design and use of technologies. This background of processes to identify a change in one's cognitive status and to alert others of such a change of their normative cognitive ability may prove helpful. This is especially important when such normative ability has been negatively affected by those who engage in physical activity involving direct and/or indirect head injuries wherein various levels of concussion may exist. A cerebral concussion essentially means that a head of a person has been subjected to external force strike or sharp pounding and the momentary obstacle of brain function that occurs. A concussion or Traumatic Brain Injury (TBI) can be mild, moderate, or severe with symptoms dependent upon the severity of the trauma. A mild TBI, or concussion, can cause an injured person to experience physical symptoms such as headache, loss of consciousness, or nausea. With moderate or severe TBI, an injured person can experience seizures or fall into a coma or a vegetative state. The severity of a TBI can be influenced by one or more factors including what part of the head was directly impacted, whether the brain moved back and forth in the skull upon impact, whether cellular structures were torn as a result of a severe rotational or spinning jolt, whether an object penetrated the skull, whether bleeding in or around the brain occurred, or the like.

It is believed that TBI is a leading cause of death and disability for America's youth. According to statistics from the Centers for Disease Control and Prevention (CDC), between one million and four million new brain injuries occur every year in America due to trauma in sports and recreational activities. More than 767,000 American youth visit the emergency room because of traumatic brain injuries each year. Of those, more than 80,000 are hospitalized and more than 11,000 die. This at-risk population is only now gaining recognition and awareness, as most youth sports activities take place with no health care professionals in attendance, and few programs are available to determine if a player may have suffered a concussion or when they should return to play after this injury. While professional sports, such as the National Football League (NFL), Major League Baseball (MLB), and National Hockey League (NHL) are implementing safety and monitoring standards, even they are utilizing only one or two of the quartet of commercially available tests which are known to be of value for adequate assessment and management of patients with TBI: physical examination by a qualified healthcare professional; neuro-cognitive testing; balance assessment; and eye movement responses. The frequency and sequelae of concussions have garnered national and international media attention, and has reached epidemic proportions, with these events now documented to be of greatest significance in younger (6- to 14-year-old) athletes whose brains have not yet become fully developed (“myelinated”). Note that sequelae of TBI include headache and dizziness, anxiety, apathy, depression, aggression, cognitive impairments, personality changes, mania, and psychosis. Typically, a sequela is a chronic condition that is a complication of an acute condition that begins during the acute condition.

Data from The Center for Disease Control (CDC) indicate that approximately every twenty-two (22) seconds someone in the United States sustains a serious traumatic brain injury. There are about 3.8 million sports and recreational related concussions in the United States every year with 1,365,000 emergency room visits and 52,000 deaths. Every year roughly fifty percent (50%) of concussions go undiagnosed. The primary reason for this is people do not realize that a potential concussion event has occurred, and therefore the injured person is not evaluated. This unfortunate, but very real problem is compounded when we learn that, for several reasons, children are much more susceptible to receiving concussions than adults. To make matters worse, when a person sustains a concussion, they are three times more likely to sustain a second concussion before the first one has had time to properly heal. Therefore, if a child has sustained even a mild concussion, and neither they nor their caregiver know, their likelihood of sustaining another dramatically increases. Unsurprisingly, the negative effects of receiving a second concussion before a first one has had time to properly heal can be significant in both the short and long term. These negative effects can be even more detrimental in children because their brains are still developing and are therefore much more sensitive to neurological injury.

Repeating sensitivity of children—Children Are at Greater Risk for Concussions than Adults. Traumatic brain injury is more common in childhood and adolescence than at any other time of life. The reasons for this are primarily due to the lack of physical development. By age 5, the skull is 90% adult size, but sits on a neck that is not as developed. The larger head sitting on a weaker neck creates a bobble-head effect that increases the chance for brain injury. Because children's brains are lighter in weight than a denser adult brain, it takes less acceleration to put a child's brain in motion when the head is suddenly stopped. The negative chemical effects that happen in the brain after receiving a concussion get started much easier in a child's brains versus an adult brain. Brain nerve fibers are coated with an insulation layer that provides strength. In children, these nerve fibers are not fully coated, making them easier to tear apar—and the tearing of nerve fibers is what helps cause a concussion.

While sports related activities account for roughly 25% of all concussions, accidental falls account for almost 50%. Unsurprisingly, the negative effects of receiving a second concussion before a first one has had Time to heal can be significant in both the short and long term. These negative effects can be even more detrimental in children because their brains are still developing and are therefore much more sensitive to neurological injury.

The improvement and problem solved as to A Process and Evaluation Method to Identify Signs of a Potential Concussion in Active Children and Others called Safe Brain to assess cognitive function that can help identify a decrease in cognitive status that may be caused by concussion should be useful. In regard to problems solved, this process: 1) uses specific steps to establish objective criteria that are used to help identify a decrease in cognitive function that may be a sign of a potential concussion, 2) implements digital alert mechanisms to notify app users that such decrease in neurological ability has occurred, indicating a potential concussion event may be in play, and 3) recommends appropriate next steps users should take when receiving such an alert.

The way in which this process differs from prior art and competition is that while they have “quick test” software to determine if someone has a decrease in brain performance or potential signs of a concussion, they only administer such test when a concussion is suspected (because an injury has been observed or is expressed from one person to another). This is a big reason roughly 50% of concussions go undetected and diagnosed. This process uses the results of interactive digital games and the cognitive assessment tool within the app and compares them against individual baselines of such games and assessment tool to indicate a decrease in cognitive ability and brain performance that may indicate the presence of injury like concussion. What people do not realize is that it does not always take a significant blow to the head for a concussion event to transpire. This new process is designed to “administer quick tests” on a regular basis as these “tests” are disguised as digital cognitive games. If someone might have a concussion based on an injury that no one observed or heard about, the interactive digital cognitive game results help to reveal a decrease in their normal brain performance for that game that was potentially caused by a concussion event. It is then that steps are initiated so the caregiver can give attention to the one that may have sustained a concussion event that otherwise would have gone undetected.

### Prior Art

A novelty search was completed for a Process and Evaluation Method to Identify Signs of a Potential Concussion in Active Children and Others called Safe Brain. It revealed the following patents in the same art:
A. Chinese Patent CN1102014742B by China in 2008 for a Field-Deployable Concussion Detector.
B. Chinese Patent CN2202010271U by China in 2011 for a Brain Concussion Treatment Table.
C. U.S. Pat. No. 9,814,426 by Conner in 2017 for a Mobile Wearable Electromagnetic Brain Activity Monitor.
D. U.S. Pat. No. 10,456,368 by Wdowin in 2019 for a Compositions for Mitigating Brain Trauma and Methods thereof.
E. US Publication 2004/0158119 by Osorio et al. in 2004 for Screening Techniques for Management of a Nervous System Disorder.
F. US Publication 2009/0191287 by Johnson in 2009 for a Mitigation of Inflammation-Related Injuries.
G. US Publication 2013/0045287 by Troup in 2013 for Treatment for Concussion Using Gallium Compounds.
H. US Publication 2013/0278899 by Waldorf et al. in 2013 for System for The Physiological Evaluation of Brain Function.
I. US Publication 2014/0330093 by Pedro in 2014 for Method for Diagnosis of And Therapy for A Subject Having a Central Nervous System Disorder.
J. US Publication 2016/0007921 by Galea et al. in 2016 for Head-Mounted Neurological Assessment System.
K. US Publication 2016/0166193 by Lin et al. in 2016 for Mobile Sensor System and Methods for Use.
L. WO 2007/016149 by Causevic et al. in 2007 for a Method for Assessing Brain Function and Portable Automatic Brain Function Assessment Apparatus/
M. WO 2018/201190 by Cohen in 2018 for Head Mountable Device.

As can be observed upon reading these referenced publications, none of the prior art has anticipated or caused one skilled in the art of identifying potential concussions to see this new process and method by Ford and Rowland as obvious or anticipated to one skilled in the industry of brain abnormalities. A Process and Evaluation Method to Identify Signs of a Potential Concussion in Active Children and Others called Safe Brain helps to identify a decrease in one's cognitive abilities and appears to be a unique and distinctive process that improves and further develops the field of measuring brain performance over time, that in turn, helps to identify potential concussions and hopefully preventing re-occurrences.

### SUMMARY OF THE INVENTION

This invention is a Process and Evaluation Method to Identify Signs of a Potential Concussion in Active Children and Others called Safe Brain. Specifically, it serves as a neurological evaluation method and process to assess cognitive function and measure brain performance over time for the purpose of revealing a change in one's cognitive status and helping to identify signs that one may have been subjected to head injury wherein various levels of concussion may exist. The Safe Brain process provides a much-needed solution to the unfortunate problem of people not realizing that a concussion event may have occurred, thus decreasing the overwhelming amount of concussions that currently go undetected. This process is done with a mobile app that also alerts children and their caregivers that such an event may have transpired. Safe Brain has designed and developed a full process and set of components used with a mobile phone that provides a much-needed solution to this growing problem. In brief, when caregivers (parents, guardians, etc) register with the app, individual neurological baselines are established for each child/person within their individual account. Once set-up, Safe Brain continues to creatively engage with children and their caregivers that enables us to provide objective criteria that indicates potential symptoms of a concussion, whether or not the child or care giver is consciously aware that an incident has occurred. This simple yet unique process of both determining and alerting children and their caregivers of a potential concussion provides a means to significantly decrease the overwhelming amount of concussions that currently go undetected.

The preferred embodiment of a Process and Evaluation Method to Identify Signs of a Potential Concussion in Active Children and Others called Safe Brain is comprised of the following steps:
Step 1: Download mobile application called “Safe Brain” onto a cell phone(s), register an account, and create individual player profiles (A, B, C) to measure each profile's individual brain performance over time to be monitored for a change in one's cognitive status.
Step 2: When able, an account administrator and/or authorized profile logs into the “Safe Brain” application.
Step 3: Each player profile (A, B, C) establishes neurological baselines using interactive digital games and a digital cognitive assessment tool called the Sign Inventory. The interactive digital games measure various cognitive functions of the player profile like memory, reaction time, coordination, etc. The digital cognitive assessment tool (SIT) is administered by an authorized caregiver that evaluates various cognitive functions typically affected by head injury like orientation, memory, sequential processing, etc. Player profile baselines are established by each profile playing the interactive digital games a certain number of times and by each profile being administered the digital cognitive assessment tool by an authorized caregiver a certain number of.
Step 4: Reminders and notifications are periodically sent to the account administrators (parents, coaches, etc) associated with the account and authorized player profiles to instruct and encourage game play several times a week;
Step 5: If a player profile's interactive digital game result falls within a certain measurable of their individual baseline, their score is used to further define their acceptable range and baseline; if the game result falls under a certain measurable of their individual baseline, an alert window appears on the screen informing them that their game result fell beneath their acceptable range and requests that they get focused and play again;
Step 6: If the player profile plays the interactive digital game again within a designated period of time and their result falls within their acceptable range, no further action transpires. However, if their game result falls under a certain measurable of their individual baseline a second time, or they do not play the game again within the designated period of time, four things occur: a) an alert window appears on the screen informing them that their game result fell below their acceptable range and that their caregiver (account administrator) has been notified, b) an alert is sent to the account administrator(s) and any other authorized party(ies) associated with the account informing them that the player profile has consecutively scored below their acceptable range in a specific game and needs attention, c) a red alert dot appears on the app icon of the smart phone indicating that a player profile within that account is in need of attention, and the player profile circle on the account's profile page turns red indicating the specific profile in the account that needs attention, and d) the player profile's game home page, viewable by the account administrator(s) is populated with an information window informing the account administrator or other authorized user to either administer the digital cognitive assessment tool (SIT) to the player profile, or to consider further evaluation for the profile.
Step 7: 1) If the caregiver elects to administer the digital cognitive assessment tool (SIT) and the result is below a certain measurable of the player profile's cognitive assessment tool (SIT) baseline, the app recommends they consider further evaluation and all red dot alerts and the profile's game home page information window remains until the player profile either a) successfully completes one of the games within their acceptable range, or b) is administered the digital cognitive assessment tool (SIT) and the result falls within their acceptable range. 2) If the caregiver elects to administer the cognitive assessment tool (SIT) and the result falls within a certain measurable of the player profile's cognitive assessment tool (SIT) baseline, the app states the player profile is within range and all red dot alerts and the profile's game home page information window go away.
Step 8: The Safe Brain application maintains a running history of each player profile's interactive digital game play results, their cognitive assessment tool (SIT) results, and when an alert notification was sent to an account administrator(s) based on results of both the player profile's digital game play results and the results of the digital cognitive assessment tool (SIT) that was administered to them;
Step 9: Repeat Steps 5 and 6 for each player profile when preparing for an upcoming physical practice or game, changing to a different type of physical activity or there is an observed change in the player profile's behavior (for example, attention patterns, sleep patterns, or apparent energy levels) or cognitive abilities.

### Objects and Advantages

There are several objects and advantages of the Process and Evaluation Method to Identify Signs of a Potential Concussion in Active Children and Others called Safe Brain. The various advantages and benefits include:

| Item | Advantages |
|---|---|
| 1 | A neurological evaluation method and process to |
| | assess cognitive function and measure brain |
| | performance over time for the purpose of revealing |
| | a change in one's cognitive status and helping to |
| | identify signs that one may have been subjected to |
| | head injury such as a concussion. |
| 2 | Most components used in the process are already |
| | available and in use, but the method and inter- |
| | workings are not used in the same manner. E.g., |
| | a set of games on a cell phone that measure |
| | various cognitive functions of the player profile |
| | like memory, reaction time, coordination, etc. |
| 3 | The method is non-threatening to the user. |
| 4 | Can be continuously updated so changes to |
| | cognitive status and brain function compared to |
| | baselines can be revealed quickly. |
| 5 | Caregivers such as parents, guardians, coaches, |
| | and others receive alerts in a real time manner |
| | informing them that normative cognitive function |
| | regarding a specific digital exercise in the app |
| | has decreased, indicating that a potential |
| | concussion may be in play. |
| 6 | The baselines and follow-up are non-invasive; no |
| | special monitoring probes or test nodes placed on |
| | the user. |
| 7 | The testing/baselines are set in a friendly |
| | environment - not at a hospital, clinic, or |
| | medical appointment, so the user is in a common, |
| | known environment. |
| 8 | The users/kids are reminded to play the cognitive |
| | digital games regularly to help maintain brain |
| | performance as a means of helping to identify |
| | cognitive decline that may be an indicator of |
| | concussion, so they may have a mild concussion |
| | that no one would have otherwise been aware of. |
| 9 | The game results versus the baselines reveal that |
| | cognitive status may be different from normal. |
| 10 | The interactive digital games that measure various |
| | cognitive functions are not intimidating to |
| | children or appear as “tests.” |
| 11 | Organizations like schools, leagues, and clubs can |
| | monitor large groups of teams and players so a |
| | trend specific to that group or sport portrays |
| | itself quickly to the caretakers and coaches. |

Finally, other advantages and additional features of the Process and Evaluation Method to Identify Signs of a Potential Concussion in Active Children and Others called Safe Brain will be more apparent from the accompanying drawings and from the full description of the device. For one skilled in the art of identifying and treating brain abnormalities such as concussions, it is readily understood that the features shown in the examples with this process can be readily adapted to other types of treatment, care and management processes, methods, and systems for concussions and brain irregularities.

### DESCRIPTION OF THE DRAWINGS-FIGURES

The accompanying drawings, which are incorporated in and constitute a part of this specification, illustrate an embodiment of the Process and Evaluation Method to Identify Signs of a Potential Concussion in Active Children and Others called Safe Brain that is preferred. The drawings together with the summary description given above and a detailed description given below serve to explain the principles of the process presented by the inventors. It is understood, however, that the method described by Ford and Rowland is not limited to only the precise arrangements, steps, and instrumentalities shown.

FIGS. 1 A through 1 D are sketches of the general Process and Evaluation Method to Identify Signs of a Potential Concussion in Active Children and Others called Safe Brain.

FIGS. 2 A through 2 H are sketches of the general Process and Evaluation Method to Identify Signs of a Potential Concussion in Active Children and Others called Safe Brain with components and features noted.

FIGS. 3 A and 3 B are the process steps and additional details of the potential concussion monitoring system known as Safe Brain.

FIG. 4 is a flowchart of the Process and Evaluation Method to Identify Signs of a Potential Concussion in Active Children and Others called Safe Brain.

FIGS. 5 A through 5 D are sketches of prior art in the field of monitoring and analyzing concussions to a brain of a human.

### DESCRIPTION OF THE DRAWINGS—REFERENCE NUMERALS

The following list of terminology refers to the drawings:

TABLE B
Reference numbers

| Ref # | Description |
|---|---|
| 30 | a Process and Evaluation Method 30 to |
| | Identify Signs of a Potential Concussions in |
| | Active Children and Others called Safe Brain 39 |
| 33 | process steps 33 in the Process and |
| | Evaluation Method 30 |
| 35 | a flowchart 35 of the Process and Evaluation |
| | Method 30 called Safe Brain |
| 36 | tracking data and baselines associated with |
| | concussions 36A, monitor concussion data 36B, |
| | and refer a person 60 with signs of a |
| | potential concussion event 77 to a medical |
| | professional 70 of necessary after data |
| | indicates a possible concussion 50 or brain |
| | trauma from a concussion event 77 such as an |
| | accident, fall, or injury in a sporting or |
| | physical activity at work or play |
| 39 | Safe Brain 39 the title of the Process and |
| | Evaluation Method 30 |
| 55 | Cognitive assessment tool 55 |
| 59 | ROCKIT 59 a skill application/game to help |
| | determine focus and short-term memory, |
| | comparing user skills against a gyroscope |
| | action of the game thus establishing another |
| | comparison point for the overall Process and |
| | Evaluation Method 30 |
| 60 | user/person 60 with a cellphone 65 setting |
| | baseline levels 63 for the Safe Brain system |
| | 30 by participating in “games” such as Brain |
| | Bash 61 or Recall 62 |
| 61 | Brain Bash 61 |
| 62 | Recall 62 |
| 63 | setting 63 baseline levels 64 |
| 64 | baseline levels 64 |
| 64 | ROCKIT 64 a skill application/game to help |
| | determine focus and short-term memory, |
| | comparing user skills against a gyroscope |
| | action of the game thus establishing another |
| | comparison point for the overall Process and |
| | Evaluation Method 30 |
| 65 | cellphone 65 with smart features such as |
| | wireless connection to the internet 110, |
| | telephone, cameras, texting, multimedia |
| | applications and the like |
| 66 | caregiver such as a parent, guardian, coach, |
| | teacher, or other 66 overseeing or monitoring |
| | or concerned about an individual a person 60 |
| | that may have suffered a concussion event 77 |
| 67 | tracking data 67 by graphs, charts, scatter |
| | diagrams or the like for presenting data and |
| | trends for data |
| 68 | referring 68 an individual 60 to a medical |
| | professional 69 |
| 69 | monitoring 69 data concerning concussions 77 |
| 70 | medical professional 70 |
| 75 | young basketball players 75 |
| 76 | young track participants 76 |
| 77 | a concussion 77 or brain trauma event |
| 78 | a football game with young, high school aged |
| | players 78 |
| 79 | young children 79 climbing playground |
| | equipment |
| 90 | downloading an application 90 for use on a |
| | cellular telephone or other mobile device 65 |
| | with smart features and ability to |
| | communicate to the internet 110 user 60 |
| 100 | prior Art 100 US Publication 2002/0091335 |
| | BRAIN FUNCTION SCAN SYSTEM by John et al. |
| 101 | prior Art 101 US Publication US 2016/0007921 |
| | HEAD-MOUNTED NEUROLOGICAL ASSESSMENT SYSTEM |
| | by Galea et al U.S. Pat. No. 1,055,416 |
| 102 | prior Art 102 WO 2018/201190 HEAD MOUNTABLE |
| | DEVICE |
| 103 | prior Art 103 WO 2007/016149 METHOD FOR ASSESSING |
| | BRAIN FUNCTION AND PORTABLE AUTOMATIC BRAIN |
| | FUNCTION ASSESSMENT APPARATUS - by CAUSEVIC |
| 110 | internet communication system 110 |
| 111 | transmission tower 111 commonly called cell |
| | towers for relaying data and communication |
| | bits on the internet 110 |
| 112 | satellites 112 as part of the data |
| | transmission for the internet 111 and |
| | cellular telephones 65 |
| 115 | data transmission waves 115 and the like as |
| | part of the internet 111 communication |
| 120 | electronic communication components 120 - |
| | computers, tablets, laptops, smart devices |
| | for manipulating data |

### DETAILED DESCRIPTION OF PREFERRED EMBODIMENT

The present development is a neurological assessment system that provides a Process and Evaluation Method to Identify Signs of a Potential Concussion in Active Children and Others called Safe Brain to assess cognitive function and measure brain performance over time for the purpose of revealing a change in one's cognitive status and helping to identify signs that one may have been subjected to head injury wherein various levels of concussion may exist.

The advantages for the Process and Evaluation Method to Identify Signs of a Potential Concussion in Active Children and Others called Safe Brain 30 are listed above in the introduction. Succinctly, the benefits are that the device:
A. A neurological evaluation method and process to assess cognitive function and measure brain performance over time for the purpose of revealing a change in one's cognitive status and helping to identify signs that one may have been subjected to head injury such as a concussion;
B. Most components used in the process are already available and in use, but the method and inter-workings are not used in the same manner. E.g., a set of games on a cell phone that measure various cognitive functions like memory, reaction time, coordination, etc.
C. The method is non-threatening to the user;
D. Can be continuously updated so changes to cognitive status and brain function compared to baselines can be revealed quickly;
E. Caregivers such as parents, guardians, coaches, and others receive alerts in a real time manner informing them that normative cognitive function regarding a specific digital exercise in the app has decreased, indicating that a potential concussion may be in play;
F. The baselines and follow-up are non-invasive; no special monitoring probes or test nodes placed on the user;
G. The testing/baselines are set in a friendly environment—not at a hospital, clinic, or medical appointment, so the user is in a common, known environment;
H. The users/kids/participants are reminded to play the cognitive digital games regularly to help maintain brain performance as a means of helping to identify cognitive decline that may be an indicator of concussion, so they may have a mild concussion that no one would have otherwise been aware of;
I. The game results versus the baselines reveal that cognitive status may be different from normal;
J. The interactive digital games that measure various cognitive functions are not intimidating to children or appear as “tests”; and
K. Organizations like schools, leagues, and clubs can monitor large groups of teams and players so a trend specific to that group or sport portrays itself quickly to the caretakers and coaches.

The preferred embodiment of the Process and Evaluation Method to Identify Signs of a Potential Concussion in Active Children and Others called Safe Brain comprised of the following steps:
Step 1: Download mobile application called “Safe Brain” onto a cell phone(s), register an account, and create individual player profiles (A, B, C) to measure each profile's individual brain performance over time to be monitored for a change in one's cognitive status.
Step 2: When able, an account administrator and/or authorized profile logs into the “Safe Brain” application.
Step 3: Each player profile (A, B, C) establishes neurological baselines using interactive digital games and a digital cognitive assessment tool called the Sign Inventory. The interactive digital games measure various cognitive functions of the player profile like memory, reaction time, coordination, etc. The digital cognitive assessment tool (SIT) is administered by an authorized caregiver that evaluates various cognitive functions typically affected by head injury like orientation, memory, sequential processing, etc. Player profile baselines are established by each profile playing the interactive digital games a certain number of times and by each profile being administered the digital cognitive assessment tool by an authorized caregiver a certain number of.
Step 4: Reminders and notifications are periodically sent to the account administrators (parents, coaches, etc) associated with the account and authorized player profiles to instruct and encourage game play several times a week;
Step 5: If a player profile's interactive digital game result falls within a certain measurable of their individual baseline, their score is used to further define their acceptable range and baseline; if the game result falls under a certain measurable of their individual baseline, an alert window appears on the screen informing them that their game result fell beneath their acceptable range and requests that they get focused and play again;
Step 6: If the player profile plays the interactive digital game again within a designated period of time and their result falls within their acceptable range, no further action transpires. However, if their game result falls under a certain measurable of their individual baseline a second time, or they do not play the game again within the designated period of time, four things occur: a) an alert window appears on the screen informing them that their game result fell below their acceptable range and that their caregiver (account administrator) has been notified, b) an alert is sent to the account administrator(s) and any other authorized party(ies) associated with the account informing them that the player profile has consecutively scored below their acceptable range in a specific game and needs attention, c) a red alert dot appears on the app icon of the smart phone indicating that a player profile within that account is in need of attention, and the player profile circle on the account's profile page turns red indicating the specific profile in the account that needs attention, and d) the player profile's game home page, viewable by the account administrator(s) is populated with an information window informing the account administrator or other authorized user to either administer the digital cognitive assessment tool (SIT) to the player profile, or to consider further evaluation for the profile.
Step 7: 1) If the caregiver elects to administer the digital cognitive assessment tool (SIT) and the result is below a certain measurable of the player profile's cognitive assessment tool (SIT) baseline, the app recommends they consider further evaluation and all red dot alerts and the profile's game home page information window remains until the player profile either a) successfully completes one of the games within their acceptable range, or b) is administered the digital cognitive assessment tool (SIT) and the result falls within their acceptable range. 2) If the caregiver elects to administer the cognitive assessment tool (SIT) and the result falls within a certain measurable of the player profile's cognitive assessment tool (SIT) baseline, the app states the player profile is within range and all red dot alerts and the profile's game home page information window go away.
Step 8: The Safe Brain application maintains a running history of each player profile's interactive digital game play results, their cognitive assessment tool (SIT) results, and when an alert notification was sent to an account administrator(s) based on results of both the player profile's digital game play results and the results of the digital cognitive assessment tool (SIT) that was administered to them;
Step 9: Repeat Steps 5 and 6 for each player profile when preparing for an upcoming physical practice or game, changing to a different type of physical activity or there is an observed change in the player profile's behavior (for example, attention patterns, sleep patterns, or apparent energy levels) or cognitive abilities.

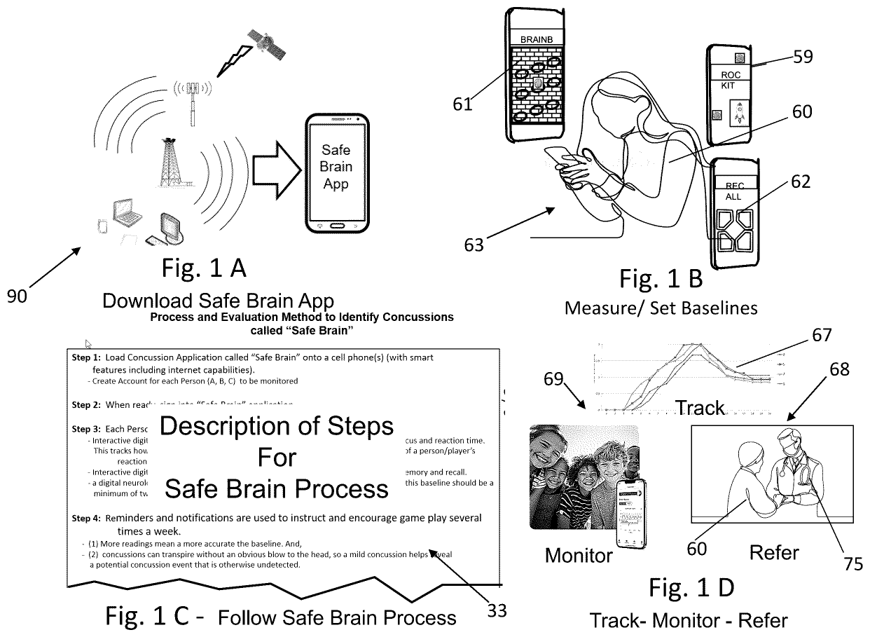

There is shown in FIGS. 1-5 a complete description and operative embodiment of the Process and Evaluation Method to Identify Signs of a Potential Concussion in Active Children and Others called Safe Brain. In the drawings and illustrations, one notes well that FIGS. 1-5 demonstrate the general configuration and use of this product. The various example uses are in the operation and use section, below.

The accompanying drawings, which are incorporated in and constitute a part of this specification, illustrate an embodiment of the Process and Evaluation Method 30 to Identify Signs of a Potential Concussion in Active Children and Others called Safe Brain that is preferred. The drawings together with the summary description given above and a detailed description given below serve to explain the principles of the Safe Brain process 30. It is understood, however, that the process 30 is not limited to only the precise arrangements and instrumentalities shown. Other examples of potential concussion evaluation and treatment processes are still understood by one skilled in the art of identifying concussion signs to be within the scope and spirit shown here.

FIGS. 1 A through 1 D are sketches of the general Process and Evaluation Method 30 to Identify Signs of a Potential Concussion in Active Children and Others called Safe Brain. Shown here are: a Process and Evaluation Method 30 to Identify Signs of a Potential Concussion in Active Children and Others called Safe Brain 39; a process steps 33 in the Process and Evaluation Method 30; a user/person 60 with a cellphone 65 setting baseline levels 63 for the Safe Brain system 30 by participating in “games” such as Brain Bash 61 or Recall 62; a setting 63 baseline levels 64; a tracking data 67 by line graphs, bar charts, scatter diagrams or the like for presenting data and trends for data; a referring 68 an individual 60 to a medical professional 70; a monitoring 69 data concerning concussions 77; a medical professional 70; and a downloading an application 90 for use on a cellular telephone 65 with smart features and ability to communicate to the internet 110 user 60.

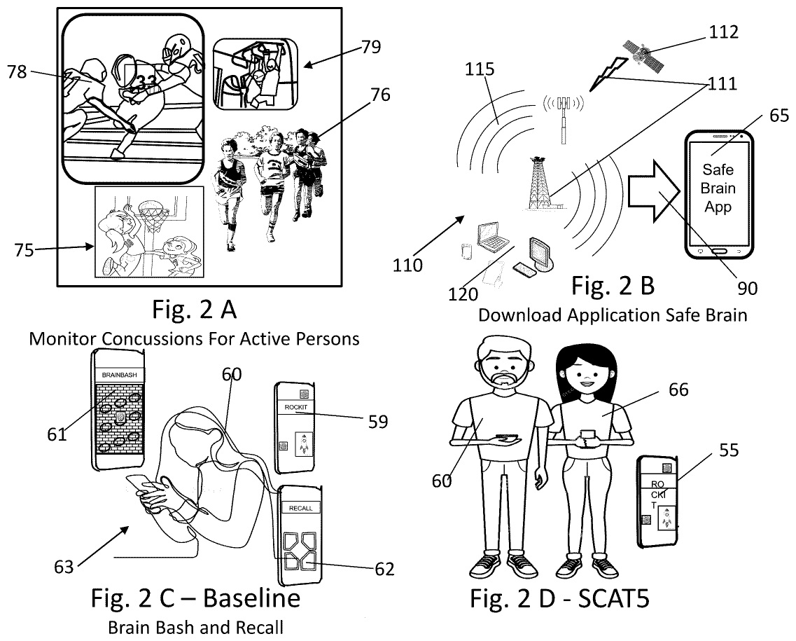

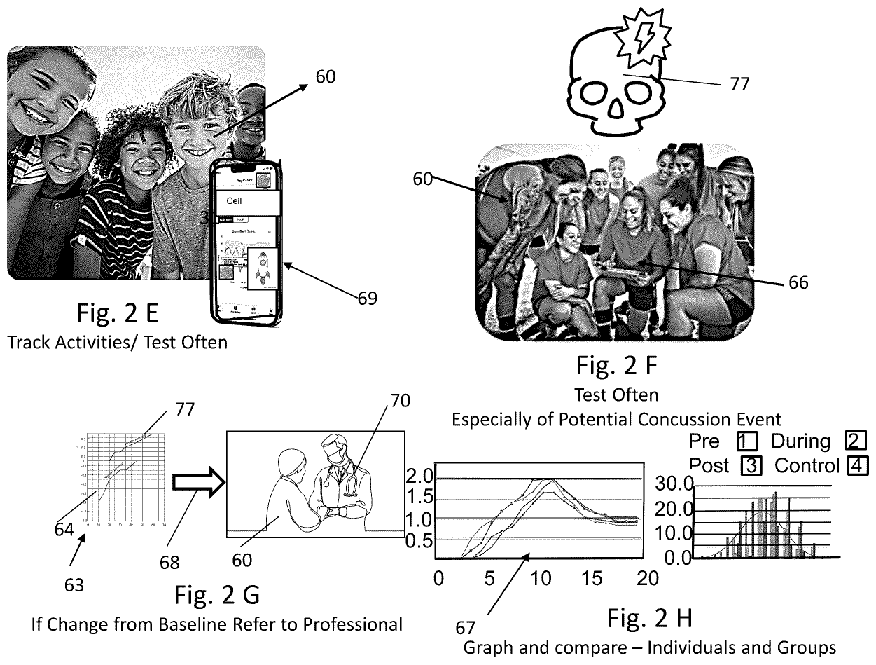

FIGS. 2 A through 2 H are sketches of the general Process and Evaluation Method 30 to Identify Signs of a Potential Concussion in Active Children and Others called Safe Brain with components and features noted. Demonstrated in these are the various components used in the Safe Brain process 30 including: a Process and Evaluation Method 30 to Identify Signs of a Potential Concussion in Active Children and Others called Safe Brain 39; a tracking data and baselines associated with concussions 36A, monitor concussion data 36B, and refer a person 60 with signs of a concussion event 77 to a medical professional 70 of necessary after data indicates a possible concussion 50 or brain trauma from a concussion event 77 such as an accident, fall, or injury in a sporting or physical activity at work or play; a Safe Brain 39 the title of the Process and Evaluation Method 30; a cognitive assessment tool neurological test, 55 called the Sign Inventory Tool (SIT); ROCKIT 59 a skill application/game to help determine focus and short term memory, comparing user skills against a gyroscope action of the game thus establishing another comparison point for the overall Process and Evaluation Method 30; a user/person 60 with a cellphone 65 setting baseline levels 63 for the Safe Brain system 30 by participating in “games” such as Brain Bash 61 or Recall 62; a Brain Bash 61; a Recall 62; a setting 63 baseline levels 64; a cellphone 65 with smart features such as wireless connection to the internet 110, telephone, cameras, texting, multimedia applications and the like; a caregiver such as a parent, guardian, coach, teacher, or other 66 overseeing or monitoring or concerned about an individual a person 60 that may have suffered a concussion event 77; a tracking data 67 by line graphs, bar charts, scatter diagrams or the like for presenting data and trends for data; a referring 68 an individual 60 to a medical professional 69; a monitoring 69 data concerning concussions 77; a medical professional 70; a concussion 77 or brain trauma event; a downloading an application 90 for use on a cellular telephone 65 with smart features and ability to communicate to the internet 110 user 60; an internet communication system 110; a transmission tower 111 commonly called cell towers for relaying data and communication bits on the internet 110; a satellites 112 as part of the data transmission for the internet 111 and cellular telephones 65; a data transmission waves 115 and the like as part of the internet 111 communication; and an electronic communication components 120—computers, tablets, laptops, smart devices for manipulating data.

Safe Brain 30 was built with industry recognized concussion specialists, providing the needed expertise to identify potential concussion signs in a new and creative way using digital game play. Brain Bash 61 and Recall 62 are Fun and Simple Games. Based on famous carnival games, Brain Bash 61 measures a child's focus and reaction time. Brain Bash 61 is based on the famous carnival game “Whac-A-Mole”. There are nine holes and brains pop up randomly one at a time during the duration of the game, and the player tries to bash as many brains as they can before the game ends. Brains are only able to be tapped for a period before going back in the hole; when one is tapped, another pops up somewhere else.

Recall utilizes a classic electronic game format to measure individual short-term memory. Recall 62 is based on the famous electronic memory game from the 80s called Simon. It presents a pattern, and the player must repeat the pattern by tapping the color tiles in the proper sequence that was just displayed to them. Each round the pattern grows one additional color pattern and the player repeats it. If they tap out of sequence the game ends. Rockit utilizes the smart phone's gyroscope feature in a creative and fun way to assess hand-eye coordination.

Baseline 63 Individual Profiles are created for each individual Person or Child to be monitored. Every person 60 is unique and has unique cognitive abilities. Individual profiles ensure objective results are obtained, based on the profile's previously established game and SIT results.

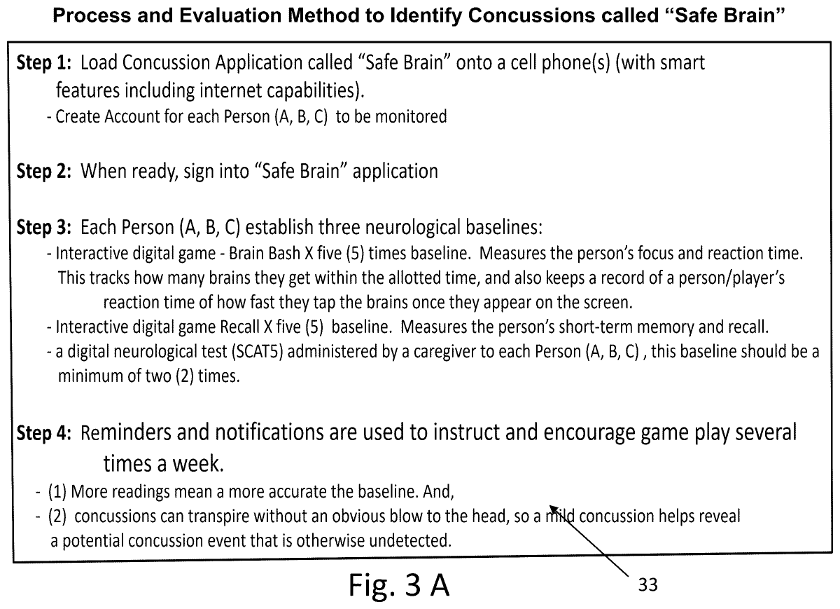

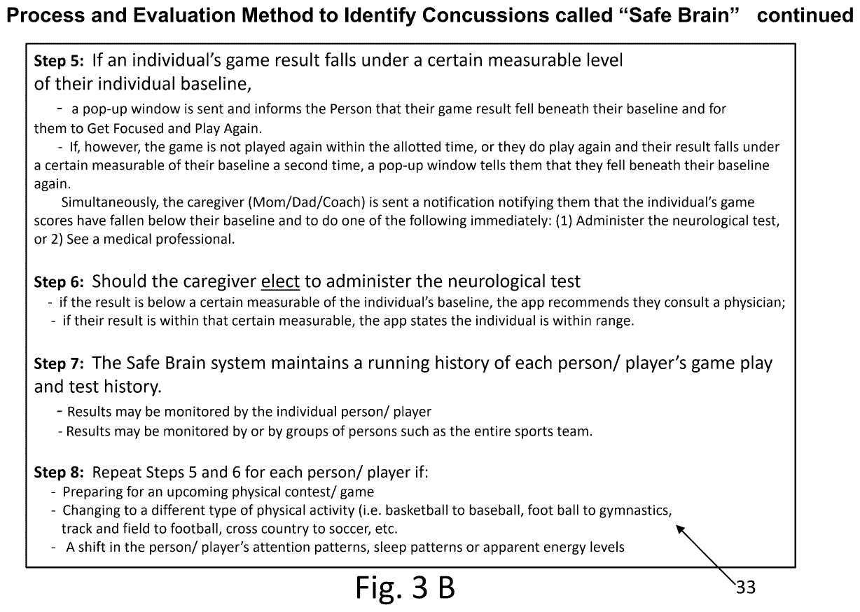

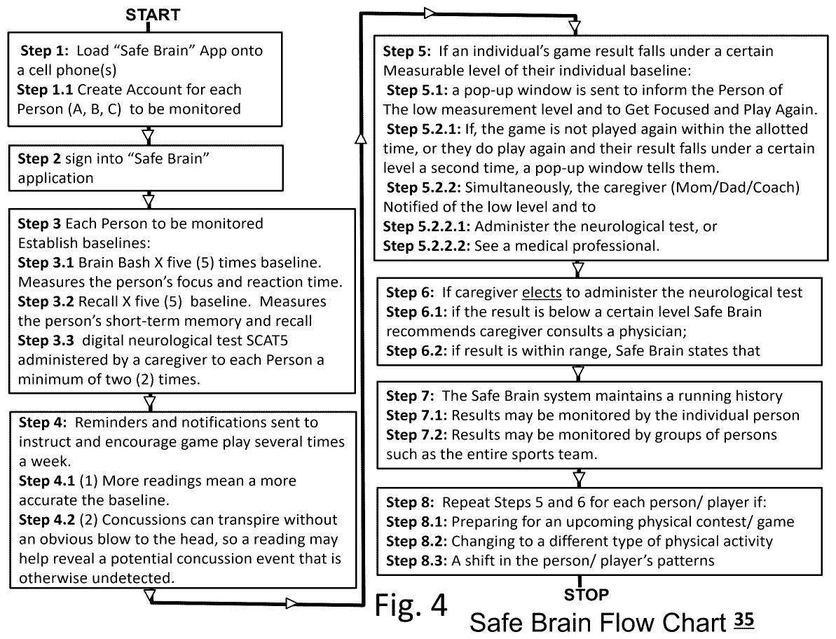

FIGS. 3 A and 3 B are the process steps and additional details of the concussion monitoring system known as Safe Brain. FIG. 4 is a flowchart of the Process and Evaluation Method to Identify Signs of a Potential Concussion in Active Children and Others called Safe Brain. These are described in the Operations Section, below.

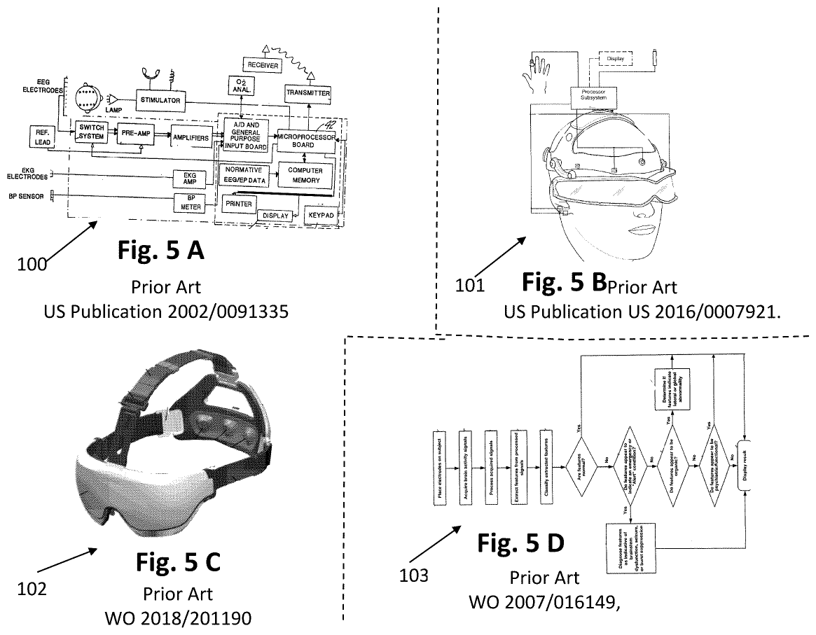

FIGS. 5 A through 5 D are sketches of prior art in the field of monitoring and analyzing concussions to a brain of a human. Here former patents and applications for various concussion process and evaluation methods for diagnosing and treating concussions. These include: Prior Art 100 US Publication 2002/0091335 BRAIN FUNCTION SCAN SYSTEM by John et al.; prior Art 101 US Publication US 2016/0007921 HEAD-MOUNTED NEUROLOGICAL ASSESSMENT SYSTEM by Galea et al U.S. Pat. No. 1,055,416; prior Art 102 WO 2018/201190 HEAD MOUNTABLE DEVICE; prior Art 103 WO 2007/016149 METHOD FOR ASSESSING BRAIN FUNCTION AND PORTABLE AUTOMATIC BRAIN FUNCTION ASSESSMENT APPARATUS—by CAUSEVIC; As can be seen, the Process and Evaluation Method to Identify Potential Signs of a Concussion in Active Children and Others called Safe Brain is a unique combination of steps and components for the purpose of revealing a change in the one's cognitive status of a person and helping to identify signs that one may have been subjected to head injury wherein various levels of concussion may exist.

The details mentioned here are exemplary and not limiting. Other specific components and manners specific to describing a Process and Evaluation Method 30 to Identify Signs of a Potential Concussion in Active Children and Others called Safe Brain may be added as a person having ordinary skill in the field of the art of diagnosing, evaluating, and treating concussions.

### Operation of the Preferred Embodiment

The Process and Evaluation Method 30 to Identify Potential Signs of a Concussion in Active Children and Others called Safe Brain has been described in the above embodiment. The manner of how Safe Brain 30 operates is described below. One notes well that the description above and the operation described here must be taken together to fully illustrate the concept of the Safe Brain method and process 30.

The Process and Evaluation Method to Identify Potential Signs of a Concussion in Active Children and Others called Safe Brain. Operates generally as follows:
1) After registering an app account, several neurological baselines are established using various interactive digital games and a digital cognitive assessment tool (the SIT). These baselines are established by the children playing the games a certain number of times and by each profile being administered the digital cognitive assessment tool by an authorized caregiver a certain number.
2) The interactive digital games measure various cognitive functions of the player profile like memory, reaction time, coordination, etc.
3) The digital cognitive assessment tool (SIT), administered by an authorized caregiver, evaluates various cognitive functions typically affected by head injury like orientation, memory, sequential processing, etc.
3) Once baselines are established, reminders and notifications are periodically sent to account administrators to instruct and encourage game play several times a week. There are two reasons for this. One, the more games are played, the more accurate the baseline becomes. Two, regular game play using the Safe Brain app helps to reveal a change in cognitive status that could indicate the presence of potential concussion event that would otherwise go undetected.
4) If an individual's game result falls under a certain measurable of their individual baseline, we send a pop-up window that informs them that their game result fell beneath their baseline and for them to get focused and play again. They have a designated amount of time to play the game again. If they play again within the designated time frame and their result falls within a certain measurable of their baseline, no further action is taken. If, however, the game is not played again within the allotted time, or they do play again and their result falls under a certain measurable of their baseline a second time, a pop-up window tells them that they fell beneath their baseline again and that their caregiver(s) have been notified. Simultaneously, the caregiver (Mom/Dad/Coach) is sent an alert notifying them that the profile's game scores have consecutively fallen below their baseline and to do one of the following immediately: (1) Administer the cognitive assessment tool (SIT), or 2) Consider further evaluation. Note that should the caregiver elect to administer the SIT and the result is below a certain measurable of the profile's SIT baseline, the app recommends they consider further evaluation; if their result is within that certain measurable, the app states the individual is within range. 5) The Safe Brain app keeps a running history of each profile game play and SIT assessment results. Note: for Brain Bash, one not only tracks how many brains they get within the allotted time, but they also keep a record of a players reaction time of how fast they tap the brains once they appear on the screen.

FIGS. 3 A and 3 B are the process steps and additional details of the concussion monitoring system known as Safe Brain. This process is:
Step 1: Downloading the “Safe Brain” mobile app onto a cell phone(s) or electronic device (with smart features including internet capabilities such as a cellphone, computers, tablets, laptops, smart devices for manipulating data). Registering an account and creating profiles (A, B, C) to be monitored.
Step 2: When able and ready, signing/logging into “Safe Brain” application by an account administrator and/or individual authorized individual.
Step 3: With each Profile (A, B, C) establishing several neurological baselines to have an individual authorized player:
Step 3.1—Interactive digital games. These various digital games like Brain Bash, Recall, and Rockit measure several different cognitive functions of the player profile like memory, reaction time, coordination, etc. Each game is played a certain number of times to establish a baseline range for each individual profile.
Step 3.2—Digital cognitive assessment tool (SIT). The SIT, administered by an authorized caregiver to each profile, evaluates various cognitive functions typically affected by head injury like orientation, memory, sequential processing, etc. The SIT is administered to each profile a certain number of times to establish the profile's SIT baseline.
Step 4: Periodically sending reminders and notifications to account administrators to instruct and encourage game play several times a week.
Step 4.1—The more often the games are played the more accurate the baseline becomes.
Step 4.2—A change in one's cognitive status can indicate that a head injury such as concussion exists; game play helps to reveal a change in a profile's cognitive status which in turn may indicate a concussion event may have occurred that would otherwise go undetected.
Step 5: A. If an interactive digital game result of an individual authorized player falls within a certain measurable of their individual baseline, their score is used to further define the acceptable range and baseline of the interactive digital game result;
B. If an individual's game result falls under a certain measurable of their individual baseline, Step 5.1—a pop-up window is sent and informs the profile that their game result fell beneath their acceptable range and for them to get focused and play again.
Step 5.2—If, however, the game is not played again within the allotted time, or they do play again and their result falls under a certain measurable of their baseline a second time, a pop-up window tells them that they fell beneath their baseline again and that their caregiver(s) have been notified.
Step 5.3—Simultaneously, the caregiver (Mom/Dad/Coach) is sent an alert notifying them that the individual's game scores have consecutively fallen below their baseline and to do one of the following c
Step 6: Should the caregiver elect to administer the SIT assessment,
Step 6.1—if the result is below a certain measurable of the profile's SIT baseline, the app recommends they consider further evaluation;
Step 6.2—if their result is within that certain measurable, the app states the profile is within range.
Step 7: The Safe Brain system maintains a running history of each profile's game play and SIT results.
Step 7.1—Results may be monitored by the profiles and account administrator(s).
Step 7.2—Results may be monitored by or by groups of persons such as the entire sports team.
Step 8: Repeat Steps 5 and 6 for each profile if: Step 8.1—Preparing for an upcoming physical contest/game.
Step 8.2—Changing to a different type of physical activity (i.e. basketball to baseball, football to gymnastics, track and field to football, cross country to soccer, etc.
Step 8.3—A shift in the person/player's attention patterns, sleep patterns or apparent energy levels.

Further discussion and information around FIGS. 3 A and 3 B as the process steps and additional details of the concussion monitoring system includes: A Process and Evaluation Method to Identify Signs of a Potential Concussion in Active Children and Others called Safe Brain which is comprised of the following steps:
Step 1: Downloading a mobile application called “Safe Brain” onto an electronic device with internet capability cell phone(s), registering an account, and creating a profile of an individual authorized player (A, B, C) wherein a change in a cognitive status in each of the individual brain performances over time can be monitored;
Step 2: When able, logging into the mobile application called “Safe Brain” by an account administrator and/or authorized individual;
Step 3: Establishing the individual authorized player for each that player's profile (A, B, C) a neurological baseline (their baseline) using a group of interactive digital games and a digital cognitive assessment tool called the Sign Inventory (SIT) which is administered by an authorized caregiver;
One noted that interactive digital games measure various cognitive functions of the player profile like memory, reaction time, coordination, etc. The digital cognitive assessment tool (SIT) is administered by an authorized caregiver that evaluates various cognitive functions typically affected by head injury like orientation, memory, sequential processing, etc.
Player profile baselines are established by each profile playing the interactive digital games a certain number of times and by each profile being administered the digital cognitive assessment tool (SIT) by an authorized caregiver a certain number of times.
Step 4: Periodically sending reminders and notifications to the caregivers administrators (parents, coaches, etc.) associated with the account and the individual authorized player profiles to instruct and encourage game play several times a week;
Step 5: A. If an interactive digital game result of an individual authorized player falls within a certain measurable of their individual baseline, using their score to further define the acceptable range and baseline of the interactive digital game result;
   B. if the game result falls under a certain measurable of their individual baseline,
   (1) alerting the individual authorized player and informing the player that their game result fell beneath their acceptable range and request that the player gets focused and play again;
   (2) alerting the caregiver to immediately Administer the SIT assessment and to Consider further evaluation; Here an alert window appears on the screen informing them that their game result fell beneath their acceptable range and requests that they get focused and play again;
Step 6: A. If the player profile is playing the interactive digital game again within a designated period of time and their result is falling within their acceptable range, no further action transpires;
   B. If the player profile is playing the game and result are falling under a certain measurable of their individual baseline a second time, or if the player profile is not playing the game again within the designated period of time, four things occur:
   (1) an alert window appears on the screen informing them that their game result fell below their acceptable range and that their caregiver has been notified,
   (2) an alert is sent to an account administrator(s) and any other authorized party(ies) associated with the account informing them that the player profile has scored below their acceptable range and needs attention,
   (3) a red alert dot appears on the app icon of the smart phone indicating that a player profile within that account is in need of attention, and the player profile circle on the account's profile page turns red indicating the specific profile in the account that needs attention, and
   (4) the player profile's game home page, is viewable by the account administrator(s) is populated with an information window and the account administrator is informed to either administer the digital cognitive assessment tool (SIT) to the individual authorized player profile, or to consider further evaluation for the profile;
Step 7: A. If the caregiver elects to administer the digital cognitive assessment tool (SIT) and the result is below a certain measurable of the individual authorized player profile's cognitive assessment tool (SIT) baseline, the app recommends they consider further evaluation and all red dot alerts and the profile's game home page information window remains until the player profile either (1) successfully completes one of the games within their acceptable range, or (2) is administered the digital cognitive assessment tool (SIT) and the result falls within their acceptable range;
   B. If the caregiver elects to administer the cognitive assessment tool (SIT) and the result falls within a certain measurable of the player profile's cognitive assessment tool (SIT) baseline, the app states the player profile is within range and all red dot alerts and the profile's game home page information window go away;
Step 8: Maintaining by the Safe Brain application a running history of each individual authorized player profile's interactive digital game play results, their cognitive assessment tool (SIT) results, and any alert notifications previously sent to an account administrator(s) based on results of both the individual authorized player profile's digital game play results and the results of the digital cognitive assessment tool (SIT) that was administered to them;
Step 9: Repeating Steps 5 and 6 for each individual authorized player profile when preparing for an upcoming physical practice or game, when changing to a different type of physical activity, or when observing a change in the individual authorized player profile's behavior. (For example, attention patterns, sleep patterns, or apparent energy levels) or cognitive abilities).

FIG. 4 is a flowchart of the Process and Evaluation Method to Identify Signs of a Potential Concussion in Active Children and Others called Safe Brain.

Step 1: Download the “Safe Brain” mobile app onto a cell phone (s)
Step 1.1 Register account and create profiles (A, B, C) to be monitored.
Step 2 Sign into “Safe Brain” application
Step 3 Each Profile (A, B, C) establishes several neurological baselines:
Step 3.1 Interactive digital games. These various digital games measure several different cognitive functions of the player profile like memory, reaction time, coordination, etc. Each game is played a certain number of times to establish a baseline range for each individual profile.
Step 3.2 Digital cognitive assessment tool (SIT). The SIT, administered by an authorized caregiver to each profile, evaluates various cognitive functions typically affected by head injury like orientation, memory, sequential processing, etc. The SIT is administered to each profile a certain number of times to establish the profile's SIT baseline.
Step 4: Reminders and notifications are periodically sent to account administrators to instruct and encourage game play several times a week.
Step 4.1 The more often the games are played the more accurate the baseline becomes.
Step 4.2 A change in one's cognitive status can indicate that a head injury such as concussion exists; game play helps to reveal a change in a profile's cognitive status which in turn may indicate a concussion event may have occurred that would otherwise go undetected.
Step 5: If an individual's game result falls under a certain measurable of their individual baseline,
Step 5.1: a pop-up window is sent and informs the profile that their game result fell beneath their acceptable range and for them to get focused and play again.
Step 5.2.1: If the game is not played again within the allotted time, or they do play again and their result falls under a certain measurable of their baseline a second time, a pop-up window tells them that they fell beneath their baseline again and that their caregiver(s) have been notified.
Step 5.2.2: Simultaneously, the caregiver (Mom/Dad/Coach) is sent an alert notifying them that the individual's game scores have consecutively fallen below their baseline and to do one of the following immediately:
Step 5.2.2.1: Administer the SIT assessment, or
Step 5.2.2.2: Consider further evaluation.
Step 6: If caregiver elects to administer the SIT assessment,
Step 6.1: if the SIT result is below a certain measureable of the profile's SIT baseline, the app recommends the profile consider further evaluation;
Step 6.2: if their result is within that certain measurable, the app states the profile is within range.
Step 7: The Safe Brain system maintains a running history of each profile's game play and SIT results.
Step 7.1: Results may be monitored by the profiles and account administrator(s).
Step 7.2: Results may be monitored by groups of persons such as coaches, trainers, athletic directors, or administrators.
Step 8: Repeat Steps 5 and 6 for each person/player if:
Step 8.1: Preparing for an upcoming physical contest/game.
Step 8.2: Changing to a different type of physical activity.
Step 8.3: A shift in the person/player's patterns.

Many events and physical activities or uses are anticipated for the Process and Evaluation Method 30 to Identify Potential Signs of a Concussion in Active Children and Others called Safe Brain. Some examples, and not limitations, are shown in the following Table.

| ITEM | DESCRIPTION |
|---|---|
| 1 | Sporting and team activities such as football, |
| | soccer, volleyball, baseball, rugby, |
| 2 | Military training and preparation |
| 3 | Individual activities such as martial arts, |
| | boxing, wrestling, |
| 4 | Vehicle activities such as bicycling, |
| | motocross, motorcycling, ATV driving, race cars |
| 5 | Individual sports like skiing, skating (ice and |
| | roller), |
| 6 | Racquet sports lacrosse, racquetball, tennis, |
| 7 | Cheerleading, acrobatics, gymnastics |

With this description it is to be understood that the Process and Evaluation Method 30 to Identify Signs of a Potential Concussion in Active Children and Others called Safe Brain is not to be limited to only the disclosed process. The steps and features of the Safe Brain process 30 are intended to cover various modifications and equivalent arrangements included within the spirit and scope of the description.

While certain novel features of this invention have been shown and described and are pointed out in the annexed claims, it is not intended to be limited to the details above, since it will be understood that various omissions, modifications, substitutions and changes in the forms and details of the device illustrated and in its operation can be made by those skilled in the art without departing in any way from the spirit of the present invention. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present inventions, the preferred methods and materials are now described above in the foregoing paragraphs.

Other embodiments of the invention are possible. Although the description above contains much specificity, these should not be construed as limiting the scope of the invention, but as merely providing illustrations of some of the presently preferred embodiments of this invention. It is also contemplated that various combinations or sub-combinations of the specific features and aspects of the embodiments may be made and still fall within the scope of the inventions. It should be understood that various features and aspects of the disclosed embodiments can be combined with or substituted for one another to form varying modes of the disclosed inventions. Thus, it is intended that the scope of at least some of the present inventions herein disclosed should not be limited by the particular disclosed embodiments described above.

The terms recited in the claims should be given their ordinary and customary meaning as determined by reference to relevant entries (e.g., definition of “plane” as a carpenter's tool would not be relevant to the use of the term “plane” when used to refer to an airplane, etc.) in dictionaries (e.g., widely used general reference dictionaries and/or relevant technical dictionaries), commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used herein in a manner more expansive than its ordinary and customary meaning, the term should be given its ordinary and customary meaning plus the additional expansive meaning, or (b) if a term has been explicitly defined to have a different meaning by reciting the term followed by the phrase “as used herein shall mean” or similar language (e.g., “herein this term means,” “as defined herein,” “for the purposes of this disclosure [the term] shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained herein should be considered a disclaimer or disavowal of claim scope. Accordingly, the subject matter recited in the claims is not coextensive with and should not be interpreted to be coextensive with any particular embodiment, feature, or combination of features shown herein. This is true even if only a single embodiment of the particular feature or combination of features is illustrated and described herein. Thus, the appended claims should be read to be given their broadest interpretation in view of the prior art and the ordinary meaning of the claim terms.

Unless otherwise indicated, all numbers or expressions, such as those expressing dimensions, physical characteristics, etc. used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques.

The present invention contemplates modifications as would occur to those skilled in the art. While the disclosure has been illustrated and described in detail in the figures and the foregoing description, the same is to be considered as illustrative and not restrictive in character, it being understood that only selected embodiments have been shown aid described and that all changes, modifications and equivalents that come within the spirit of the disclosures described heretofore and or/defined by the following claims are desired to be protected.

What is claimed is:

1. A Process and Evaluation Method to Identify Signs of a Potential Concussion in Active Children and Others called Safe Brain which is comprised of the following steps:
Step 1: Downloading a mobile application called “Safe Brain” onto an electronic device with internet capability, registering an account, and creating a profile of an individual authorized player (A, B, C) wherein a change in a cognitive status in each of the individual brain performances over time can be monitored;
Step 2: When able, logging into the mobile application called “Safe Brain” by an account administrator and/or authorized individual;
Step 3: Establishing the individual authorized player for each that player's profile (A, B, C) a neurological baseline (their baseline) using a group of interactive digital games and a digital cognitive assessment tool called the Sign Inventory (SIT) which is administered by an authorized caregiver;
Step 4: Periodically sending reminders and notifications to the caregivers and the individual authorized player profiles to instruct and encourage game play several times a week;
Step 5: A. If an interactive digital game result of an individual authorized player falls within a certain measurable of their individual baseline, using their score to further define the acceptable range and baseline of the interactive digital game result;
B. if the game result falls under a certain measurable of their individual baseline,
(1) alerting the individual authorized player and informing the player that their game result fell beneath their acceptable range and request that the player gets focused and play again, and
(2) alerting the caregiver to immediately Administer the SIT assessment and to Consider further evaluation;
Step 6: A. If the player profile is playing the interactive digital game again within a designated period of time and their result is falling within their acceptable range, no further action transpires;
B. If the player profile is playing the game and result are falling under a certain measurable of their individual baseline a second time, or if the player profile is not playing the game again within the designated period, four things occur:
(1) an alert window appears on the screen informing them that their game result fell below their acceptable range and that their caregiver has been notified,
(2) an alert is sent to an account administrator(s) and any other authorized party(ies) associated with the account informing them the player profile has scored below their acceptable range and needs attention,
(3) a red alert dot appears on the app icon of the smart phone indicating that a player profile within that account needs attention, and the player profile circle on the account's profile page turns red indicating the specific profile in the account that needs attention, and
(4) the player profile's game home page, is viewable by the account administrator(s) is populated with an information window and the account administrator is informed to either administer the digital cognitive assessment tool (SIT) to the individual authorized player profile, or to consider further evaluation for the profile;
Step 7: A. If the caregiver elects to administer the digital cognitive assessment tool (SIT) and the result is below a certain measurable of the individual authorized player profile's cognitive assessment tool (SIT) baseline, the app recommends they consider further evaluation and all red dot alerts and the profile's game home page information window remains until the player profile either (1) successfully completes one of the games within their acceptable range, or (2) is administered the digital cognitive assessment tool (SIT) and the result falls within their acceptable range;
B. If the caregiver elects to administer the cognitive assessment tool (SIT) and the result falls within a certain measurable of the player profile's cognitive assessment tool (SIT) baseline, the app states the player profile is within range and all red dot alerts and the profile's game home page information window go away;
Step 8: Maintaining by the Safe Brain application a running history of each individual authorized player profile's interactive digital game play results, their cognitive assessment tool (SIT) results, and any alert notifications previously sent to an account administrator(s) based on results of both the individual authorized player profile's digital game play results and the results of the digital cognitive assessment tool (SIT) that was administered to them; and
Step 9: Repeating Steps 5 and 6 for each individual authorized player profile when preparing for an upcoming physical practice or game, when changing to a different type of physical activity, or when observing a change in the individual authorized player profile's behavior.

2. The Process and Evaluation Method to Identify Signs of a Potential Concussion in Active Children and Others called Safe Brain in claim 1 wherein the electronic device with internet capability is selected from the group consisting of cell phones, computers, tablets, laptops, and smart devices.

3. The Process and Evaluation Method to Identify Signs of a Potential Concussion in Active Children and Others called Safe Brain in claim 1 wherein the Interactive digital games is selected from the group consisting of Brain Bash, Recall, and Rockit.

4. The Process and Evaluation Method to Identify Signs of a Potential Concussion in Active Children and Others called Safe Brain in claim 1 wherein the Interactive digital games measure several different cognitive functions of the profile of the individual authorized player including memory, reaction time, coordination.

5. The Process and Evaluation Method to Identify Signs of a Potential Concussion in Active Children and Others called Safe Brain in claim 1 wherein the digital cognitive assessment tool called the Sign Inventory (SIT) is administered by an authorized caregiver that evaluates various cognitive functions typically affected by head injury like orientation, memory, and sequential processing.

6. The Process and Evaluation Method to Identify Signs of a Potential Concussion in Active Children and Others called Safe Brain in claim 1 wherein the caretaker is
